(19) United States
(12) Patent Application Publication
(10) Pub. No.: US 2019/0078052 A1
(43) Pub. Date: Mar. 14, 2019

(54) ENGINEERED ADHESIVE SUBSTRATES FOR HIGH-THROUGHPUT CELL ISOLATION AND SEPARATION

(71) Applicant: ARIZONA BOARD OF REGENTS ON BEHALF OF ARIZONA STATE UNIVERSITY, Scottsdale, AZ (US)

(72) Inventors: Kaushal Rege, Chandler, AZ (US); Taraka Sai Pavan Grandhi, Tempe, AZ (US)

(21) Appl. No.: 16/084,415

(22) PCT Filed: Mar. 16, 2017
(86) PCT No.: PCT/US17/22675

§ 371 Date: Sep. 12, 2018

Related U.S. Application Data
(60) Provisional application No. 62/309,307, filed on Mar. 16, 2016.

(51) Int. Cl. C12N 5/00; C12N 5/09

(52) U.S. Cl. CPC C12N 5/0068; C12N 5/0693; C12N 5/0656; C12N 2533/50; C12N 5/0062

(57) ABSTRACT

Methods and compositions involving hydrogel compositions utilized for growing, separating, isolating, and/or screening cancer cells for resistance to one or more anti-cancer cell agents, such as a drug or biologic. Some hydrogel compositions utilize the monomer aminoglycoside amikacin AM1 or aminoglycoside amikacin AM3 in combination with a variety of crosslinkers.

### CROSS-REFERENCE TO RELATED APPLICATIONS

This application claims the benefit of U.S. Provisional Patent Application No. 62/309,307, filed Mar. 16, 2016, which is incorporated herein by reference as if set forth in its entirety.

### FIELD OF TECHNOLOGY

This disclosure related to substrates for cell isolation and in some embodiments to adhesive substrates for metastatic and/or drug resistant cancer cell isolation and separation.

### BACKGROUND

Tumors are heterogeneous in their genotypic and phenotypic makeup. Upon exposure to a certain anticancer drug, only the susceptible fraction of the cancer population undergoes ablation, leaving the resistant population to repopulate the tumor. Primary treatments such as chemotherapy, radiotherapy, surgery or biologic therapy that are prescribed for cancer patients work to ablate the sensitive cell population, leaving the resistant cell population behind.

Thus, it remains an ongoing challenge for researchers and clinician alike to characterize heterogeneous tumor cell populations and devise treatment strategies in view thereof.

### SUMMARY

Methods and compositions utilizing hydrogel compositions for growing, separating, isolating, and/or screening cancer cells for resistance to one or more anti-cancer cell agents, such as a drug or biologic.

Some hydrogel compositions described herein utilize the monomer aminoglycoside amikacin AM1 or aminoglycoside amikacin AM3 in combination with a variety of crosslinkers.

Accordingly, this disclosure relates to novel substrates that can directly isolate the metastatic cellular fractions from a heterogenous cancer cell population. The chemo-mechanical properties of the substrate can be modulated such that only the most metastatic and most drug resistant cellular fractions are isolated and separated.

Unlike traditional separation or isolation techniques, embodiments herein do not require the use of natural materials such as collagen, fibronectin, etc.

In some method embodiments, isolation of highly drug resistant and metastatic fractions of cancer cells can allow for further research to discover novel phenotype specific drug, biologics, immunotherapies and their combinations.

### BRIEF DESCRIPTION OF THE FIGURES

FIG. 1. Qualitative measurement of amikagel adhesivity compared to 2D tissue culture plastic indicated ˜40% lower adhesivity allowed for isolation of only the N-cadherin poor, metastatic fraction of cancer cells. Amikagel AM1 incorporated higher units of amikacin crosslinked with PEGDE (polyethylene glycol diglycidyl ether) (higher number of amikacin amines compared to the epoxide groups on the PEGDE) resulting in a crosslinked substrate whose adhesivity and mechanical stiffness are engineered to isolate the metastatic cell fractions. We show that by incorporating multiple randomly cross-linked alternating units of adhesive and non-adhesive monomers into a polymeric substrate, a novel synthetic cell isolation platform can be created. Our system directly integrates the adhesive and non-adhesive components into the matrix. Unlike other techniques, our system does not require coating with any other substance such as collagen, fibronectin, etc.

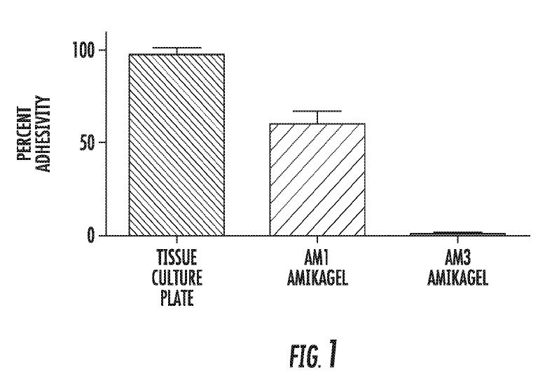

FIG. 2. Chemo-mechancial engineering of Amikagels induces selective relapse from dormancy—T24 3D-DTMs were transferred from AM3 to AM1 Amikagels and visualized for changes in morphology. Phase contrast image of the transferred 3DTM at (A) Day 0, (B). Day 1, and (C) Day 15 after transfer. Following transfer of dormant T24 3DTMs from AM3, cell shedding on AM1 resulted in the formation of microcolonies, 70-100 μm diameter, within 15 days (C). Representative images are shown (n=3). Scale bar=100 μm in all cases. (D-E) Cell cycle distribution indicated that the ‘mother’ T24 3DTM remained in near-complete arrest in the G0/G1 phase (˜90% cells in G0/G1 phase). However, cells that escape the dormant mother 3D-DTM, spread on AM and form microcolonies showed a more proliferative profile (17% cells in the G2/M phase compared to 5% G2/M cells in the mother spheroid). (F) Relapsed cells were observed to have lower N-cadherin levels (significantly lower fluorescence) compared to cells that remained dormant after relapse (Mother 3D-DTM). (G) Relapsed cells were also observed to actively consume media compared to the expanded mother 3D-DTM cells.

* indicates p-value <0.05 (n=2, Student's t-test) ** indicates p-value <0.01 (n=3, Student's t-test) for the G2/M populations of escaped cells compared to the dormant mother 3DTM, indicating an actively proliferating population in the shed cells.

FIG. 3. Docetaxel significantly reduces the relapse from tumor dormancy. (A). Experimental sequence. (B). Representative image of dormant T24 3D-DTM grown on AM3 and transferred to AM1 this 3DTM was not treated with docetaxel. Image taken after 48 hours of transfer of dormant T24 3D-DTM to AM1 gel showed significant cell escape from the dormant mother 3D-DTM with filopodia formation (black arrow). (C). Representative image of dormant T24 3D-DTM formed and subsequently treated with 100 μM docetaxel on AM3. The pre-treated 3D-DTM was then transferred to AM1. Image taken after 48 hours of transfer of the docetaxel pre-treated dormant T24 3D-DTM to AM1 gel. As seen in the picture, significantly lesser number of cells escaped the mother spheroid after pre-treatment with docetaxel. Shed cells did not show filopodia formation (black arrow). Microcolony formation in case of (D) untreated and (E) 100 μM docetaxel pre-treated T24 3D-DTMs after 15 days of transfer to AM1. Docetaxel pre-treatment significantly reduced cell escape and microcolony formation. Scale bar=100 μm in all cases. All the experiments were performed at least n=3 independent times.

FIG. 4. Cell cycle analysis of T24 3D-DTMs after 96 hours with docetaxel on AM3. (A) Cell cycle distribution of T24 3D-DTMs after treatment without and with 100 uM of docetaxel for 96 hours (M1-Pre G0/G1 phase, M3-S phase, M4-G2/M phase, M5-Multiploid cells). (B) Distribution of cells in pre-G0/G1 phases after treatment with 0 uM, 50 uM, and 100 UM docetaxel for 96 hours. N=3 independent experiments.

### DETAILED DESCRIPTION

Embodiments herein relate to compositions and methods for cell growth, separation, isolation, and/or screening. In some embodiments, the cells are metastatic and/or drug resistant cancer cells.

While epithelial cells are contact inhibited, terminally differentiated, and posses low migratory abilities, the mesenchymal phenotype of the cancer cells show no cell cycle arrest after cell-cell contact, have high migratory abilities, matrix metalloproteinases production, etc. This EMT switch has been shown to be a critical hallmark of cancer growth and metastases to secondary sites. Isolation of such metastatic fraction of the cancer population not only allows for development of drugs against those fractions, but also allow continuous monitoring of the disease towards development of any novel metastatic phenotypes.

Novel substrates have been developed that not only isolate the metastatic fraction of the cells, but also allow for their easy recovery and separation from the heterogenous cancer population.

For example, aminoglycoside amikacin was crosslinked with crosslinker PEGDE (polyethylene glycol diglycidyl ether) in different mole ratios to give a hydrogel (referred here as amikagel) of varying chemo-mechanical properties. Chemo-mechanical properties here refers to cellular adhesivity coupled to the stiffness of the gel. Aminoglycoside amikacin has 4 primary amine sites that provide adhesivity to the cells in a hydrogel substrate formulation, whereas PEG groups of the PEGDE provide non-adhesivity to the substrate. In this embodiment, amikacin hydrate has a molecular weight of ˜585 Da whereas the PEGDE has a molecular weight of ˜500. A mixture of these two monomers in different mole ratios leads to the design and development of substrates that have equivalent or non-equivalent adhesive and non-adhesive areas along the gel.

### Non-Limiting Examples

### Amikagel Synthesis

Ring-opening polymerization between amine groups of amikacin hydrate and epoxide groups of poly(ethylene glycol) diglycidyl ether (PEGDE) resulted in the formation of a novel hydrogel henceforth called ‘Amikagel’. Different stoichiometric ratios of amikacin and the cross-linker PEGDE were dissolved in Nanopure® water, mixed and incubated at 40° C. for 7.5 h, in order to obtain Amikagels AM1, AM2, and AM3 of different compositions (Table 1):

| | Molar ratio of | Volume of PEGDE added |
|---|---|---|
| Sample | amine/epoxide | (μl)/25 mg of Amikacin |
| AM1 | 1:1.5 | 28.125 |
| AM2 | 1:2 | 37.5 |
| AM3 | 1:3 | 56.25 |

The final concentration of amikacin was 10 wt % in all gels. All experiments were carried out in triplicate unless otherwise mentioned. AM1 was the most adhesive and mechanically weak, whereas AM3 was the least adhesive and mechanically strong.

### Detailed Protocol for Specific Cell Isolation—Step 1 of Isolation—

1 ml of amikagel AM1, AM2 and AM3 pre-gel solutions were filtered through a 0.20 μm filter and 40 μL of the filtrate was added to each well of a 96 well plate. The plates were sealed with paraffin tape (Parafilm, Menasha, Wis.) and incubated in an oven maintained at 40° C. for 7.5 hours. After gelation, the surfaces of Amikagels were washed with 150 μL of Nanopure® water for 12 hours, in order to remove traces of unreacted monomers.

All 3DTM (3D tumor microenvironments) experiments were set up by liquid overlay culture (2) of cells on top of Amikagel surface in a total volume of 150 L media/well; either 100,000 cancer cells alone (single culture) or 50,000 stromal cells followed by 50,000 cancer cells (co-culture) were incubated, unless indicated otherwise in specific cases. After 48 hours of incubation, 50% of the media in the wells was replaced with fresh media i.e. DMEM/RPMI+10% (v/v) FBS+1% (v/v) Pen-Strep at regular intervals of 48 hours. Care was taken to withdraw and add the media slowly so as to not perturb 3DTM formation. Fresh media was added every 48 hours following cell plating. For 3DTM generation on 24 well plates, 400 μL of pre-gel volume was used instead of 40 μl. Different co-culture 3DTM systems are represented as fibroblast/stromal cells-epithelial cells (e.g. NIH3T3-T24, WPMY-1-T24) to accurately indicate the sequence of their addition. In most cases, 3DTMs were formed 5-7 days following culture on Amikagels.

Step 2 of Cell Isolation: Transfer of 3DTMs from AM3 Amikagel to Chemo-Mechanically Engineered AM1 Gel

T24 3DTMs were first formed on AM3, and transferred to AM1s on the seventh day following initial cell seeding, in order to investigate the role of chemo-mechanical properties of Amikagels on 3DTM fate, and migration of metastatic cells out of the 3DTM spheroid. Upon transfer, cells from 3DTMs were monitored for cell spreading and motility on the gel for an additional 7 days. After 7 days, cell cycle analysis and N-cadherin analysis was carried out on the all 3DTMs. Long-term experiments were also carried out where 3D-DTMs were continuously monitored for 15 days after their transfer from AM3 gel to AM1 gel.

### Cell Cycle Analyses

Following five days of incubation on AM3, 3DTMs of T24 cells with NIH3T3 murine fibroblast cells were harvested for cell cycle analysis. Four or five individual 3DTMs of T24 cells alone or UMUC3 cells alone were harvested on the 7th day after seeding on Amikagels, collected in an eppendorf tube. 50 μL of 5 mg/ml collagenase was added to 3DTMs prepared using fibroblast helper cells for 30 minutes at 37° C. in order to facilitate their disassembly by gentle pipetting. Single cell 3DTMs were disassembled using manual pipetting.

Disassembled 3DTM cells were then centrifuged at 200 r.c.f. in order to collect the cell pellet. The pellet was resuspended in a solution of 1% v/v 1× Triton-X, 5% (v/v) fetal bovine serum (FBS), 50 μg/mL propidium iodide, and 0.006-0.01 units/mL ribonuclease A. After incubation for 30 minutes on ice, cells were analyzed for their cell cycle profiles using flow cytometry; the propidium iodide (PI) signal was detected using an excitation at 535 nm and emission at 617 nm.

Voltages of the FL2-A, SSC and FSC channels were adjusted in order to obtain best representative peaks for alignment of 2n (diploidy−G0/G1) peak to 200 intensity units during flow cytometry. FL2A (FL2-Area) provides the information regarding the pulse area of the emitted fluorescence signal (total cell fluorescence) whereas SSC and FSC provide the information regarding the forward scatter and the side scatter light from the sample. FSC is a measure of diffracted light from the sample proportional to cell surface area or size and SSC is proportional to cell granularity or internal complexity.

N-Cadherin Expression on Relapsed and Dormant after Relapse Cells on AM1

After 15 days of transfer of T24 3D-DTM to AM1, the relapsed cells and the remnant mother 3D-DTM were collected and expanded on fresh 2D cell culture plates. After 48 hours of expansion, 600,000 cells of the two cell populations were collected for N-cadherin surface expression studies. Briefly, the cells were detached from the surface using 20 mM EDTA in ice-cold 1×PBS. After 30 minutes of rocking at 4° C., the cells were collected and blocked with wash buffer (1×PBS containing 2% FBS) for 30 minutes at 4° C. Wash buffer and block buffer were composed of 1×PBS containing 2% FBS. After 30 minutes of washing, the cells were incubated with primary antibody at a concentration of 20 μg/mL in 1×PBS containing 2% FBS at 4° C. for 1 hour under gentle rocking. The cells were collected by centrifugation and washed three times, five minutes each in ice cold wash buffer. The anti-mouse secondary antibody conjugated with Alexa-488 was added to the cells at a dilution of 1:200 for 30 minutes in 1×PBS containing 2% FBS at 4° C. followed by three washes. Flow cytometry was performed as described before. N-cadherin expression on cell populations was expressed as mean fluorescent peak.

### Statistical Analyses

Averages have been expressed as mean±SD. The effectiveness of the drug combinations were quantified using the combination index (CI) by Chou-Talalay method. Two-tailed t-test with 95% CI was used analyze and compare the percent cell death data of individual drugs. One-way ANOVA has been used to study the differences between the effectiveness of multiple drugs and their combinations. Tukey's multiple comparisons test was used during multiple pairwise comparisons whereas Dunnett's multiple comparisons test was used while comparing multiple means to a single one (control). p<0.05 indicated significance in the analyses. All analyses were performed using the Prism GraphPad software. All experiments have been performed at least n=2 or more independent times with three replicates each unless specified.

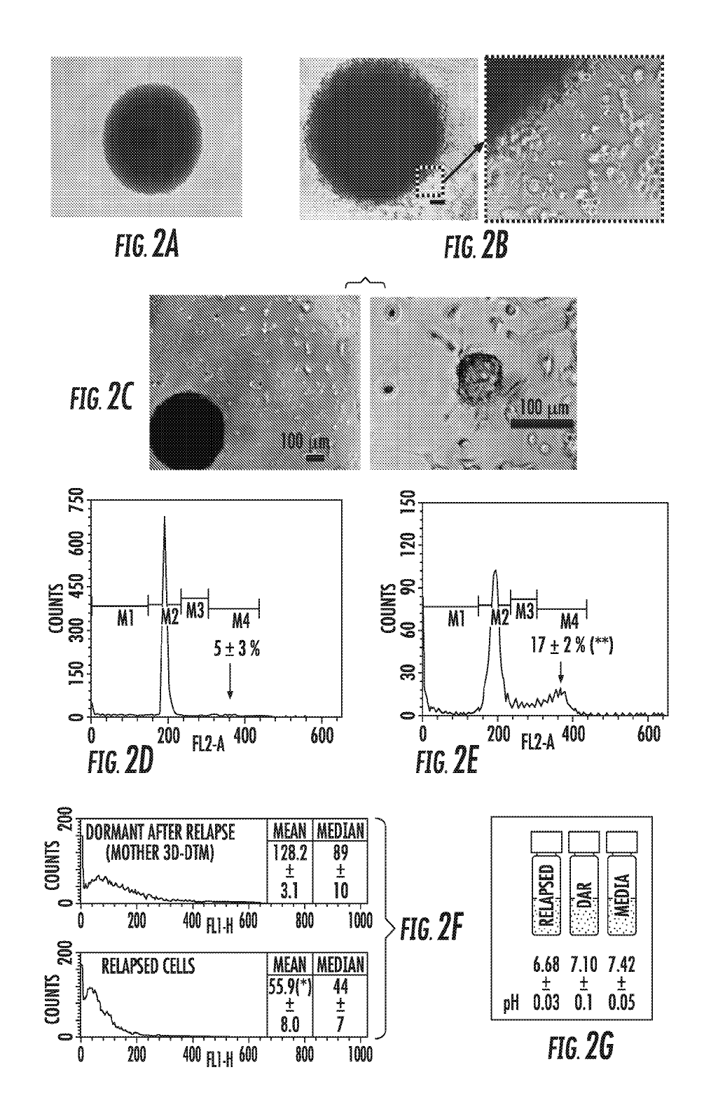

T24 3D-DTMs, generated on mechanically stiff and non-adhesive AM3, were transferred to more adhesive but mechanically weaker AM1, in order to model changes in the tumor microenvironment. T24 cells escaped from the ‘mother 3D-DTM’ within just 24 hours following transfer to AM1 (FIG. 2A-B). However, no cell escape was observed when 3D-DTMs generated on AM3 were transferred onto freshly prepared AM3 instead of AM1, indicating that the different chemomechanical microenvironment played a key role in escape of cells. At 15 days following transfer, it was clear that not all cells had left the mother 3D-DTM placed on AM1.

Interestingly, cells that escaped formed micrometastasis-like nodules, 70-100 μm in diameter, on AM1 at significant distances away from the mother 3D-DTM (FIG. 2C). Cell cycle studies, seven days following transfer, indicated that the ‘mother 3D-DTM’ continued to remain dormant (FIG. 2D), while the shed cells (FIG. 2E) demonstrated increased number of proliferating cells (FIG. 2D-E).

We studied the N-cadherin expression on the expanded populations of the mother 3D-DTM and the relapsed cells and found significant differences between them. N-cadherin expression was almost 50% lower in the relapsed cells compared to the cells that remained dormant after relapse (Mother 3D-DTM) (FIG. 2F). Changes in media color was further indicative of active metabolism and proliferation in case of shed cells on AM1, indicating a reversal of these cells from a dormant to proliferative phenotype compared to the mother 3D-DTM (FIG. 2G). T24 cell line is known to be heterogeneous with a mix of metastatic and non-metastatic cell fractions. Low N-cadherin has been associated with significantly poor prognosis and accelerated death in bladder cancer.

Modulating Amikagel's adhesivity allowed for selective migration, isolation and easy recovery of N-cadherin poor population of T24 cells. Highly adhesive substrates such as 2D tissue culture plate caused total invasion and substrate integration of the 3D-DTM, making the recovery difficult (not shown). Amikagel's adhesivity was found to be ˜40% lower than 2D tissue culture plate and hence made it easier only for metastatic cells to escape. Taken together, modulating chemo-mechanical properties of Amikagels resulted in 3D models of (1) tumor dormancy, (2) cellular escape from dormancy, (3) formation of micrometastasis-like nodules, and (4) selective isolation of highly metastatic cell fractions using a single platform.

Taking a cue from bladder cancer escape and metastasis following ECM mechanical changes, we show that chemo-mechanical modulation of Amikagel was able to engender relapse of certain cancer cells from dormancy. The relapsed cells demonstrated a proliferative phenotype, with lower N-cadherin levels and a some of these formed micrometastasis-like colonies on the gel. The tumorigenic variant of T24 cells formed microcolonies on soft agar and it has been suggested a paracrine signaling pathway of communication between these cells is activated upon mutual contact. These cells also had higher expression of HRAS, lower expression of β-catenin that led to focal adhesion disassembly and invasion. T24 cells are known to be mesenchymal-like, E-cadherin null and likely heterogeneous N-cadherin expression, which makes our selective, heterogeneous cell escape and subsequent microcolony formation results unique. By modulating the adhesivity of the substrate, Amikagel could induce the migration of only the most metastatic, N-cadherin poor cells, allowing for easy separation and recovery unlike 2D tissue culture plastic.

Modulation of Amikagel chemomechanical properties likely facilitated the separation of this heterogeneous population into N-cadherin rich dormant and N-cadherin poor relapsed and micrometastases-like colony forming cells. While N-cadherin is a significant prognostic factor in bladder cancer progression, reduction of N-cadherin was found to be associated with enhanced patient mortality rates. Selective and easy substrate assisted isolation and recovery of N-cadherin poor metastatic cells significantly improves the clinical relevance of Amikagels in bladder cancer. Chemo-mechanical biomaterial strategies could allow for engineering substrates that directly isolate the most metastatic cell types, rather than doing so repeatedly in the mice.

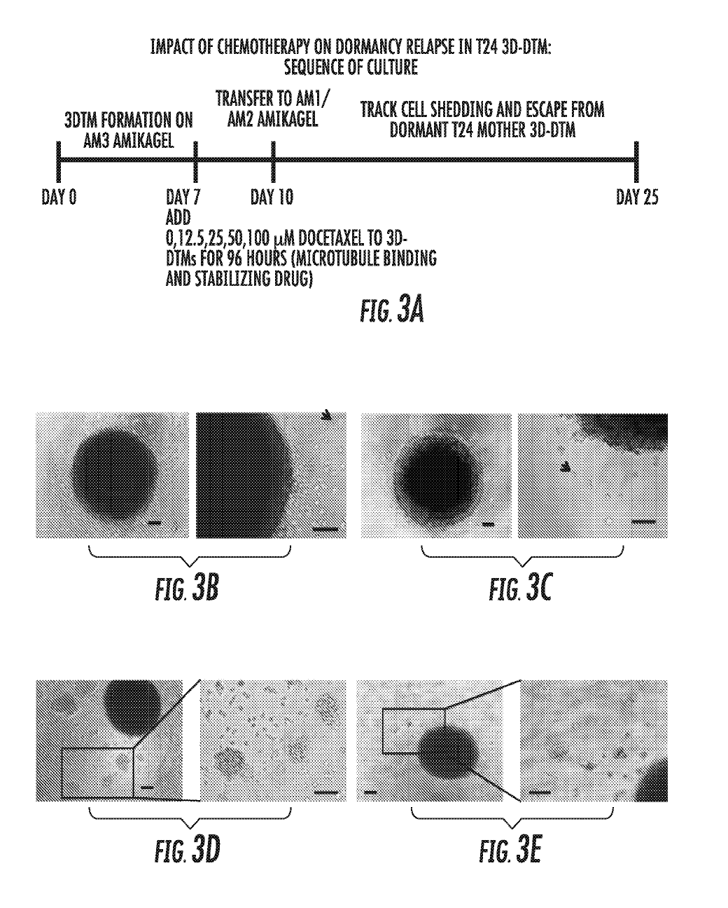

Docetaxel treatment (12.5 μM-100 μM) significantly reduced cellular escape from the mother 3D-DTM (FIG. 3A-C), likely due to its ability to inhibit cell migration. Prior research indicated that docetaxel reduced the expression of phospho-AKT and phospho-FAK by approximately ˜41% and ˜34% respectively compared to untreated T24 cells; both AKT and FAK are involved in regulating bladder cancer cell migration. In addition, docetaxel has also been shown to effectively inhibit cdc42, which promotes formation of actin-rich filopodia and their extension prior to cell migration in other cancer cell lines. Filopodial extensions were not observed on cells shed on AM1 after T24 3D-DTMs docetaxel treatment (FIG. 3B-C, Black arrows).

Formation of micrometastasis-like nodules was also drastically reduced following docetaxel-treatment, while untreated 3D-DTMs continued to demonstrate formation of these microcolonies (FIG. 3D-E). T24-3D-DTMs treated with docetaxel remained viable and showed a dormant cell cycle profile following treatment, indicating that reduction of cell escape from dormancy is not due to cell death.

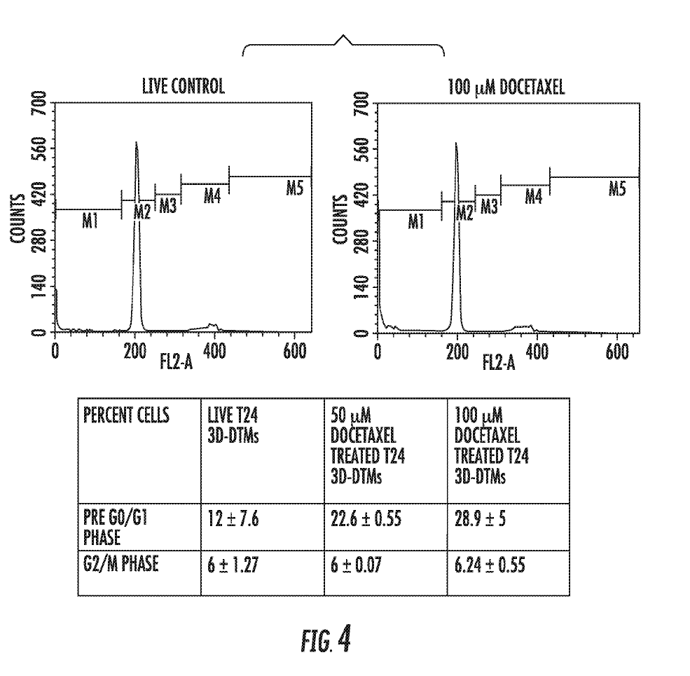

Cell cycle distribution of docetaxel-treated mother 3D-DTM (FIG. 4 C-D) showed a modest increase of cells in the sub-G0/G1 phase of the cell cycle. This indicates a slight increase in the number of cells undergoing apoptosis, which is consistent with previous cell viability results observed with docetaxel. No significant differences were observed in cells in the G2/M phase of the cell cycle between the untreated 3D-DTM and docetaxel-treated 3D-DTM (FIG. 4C-D). However, the escape of some cells from the mother 3D-DTM after docetaxel treatment and insignificant changes in the viability of the 3DTM are indicative of the challenges in restricting tumors to a dormant state when microenvironment conditions eventually change (e.g. change in adhesivity and/or mechanical properties as in case of transfer from AM3 to AM1). Isolation of the cells that migrate out of the 3D-DTM after docetaxel treatment are the ones that retain cell viability and migratory abilities even after drug exposure. These cell fractions are the most metastatic and are the ones that will likely survive the chemotherapeutic insult. Chemo-mechanical engineering of Amikagel allowed for isolation of specific cell fractions that are not only highly drug resistant, but also retain migratory and metastatic abilities after drug exposure.

Examples of substrates include, but are not limited to, the following. Adhesive components—aminoglycosides—streptomycin, neomycin, framycetin, paromomycin, ribostamycin, kanamycin, arbekacin, bekanamycin, dibekacin, tobranmycin, spectinomycin, hygromycin b, gentamicin, netilmicin, sisomicin, isepamicin, verdamicin, astromicin, apramycin or any other amine or hydroxyl rich moieties, such as collagen, fibronectin, laminin, extracellular matrix, fibrin, short peptides, RGD peptide, polyethyleneimine, oligonucleotides, aptamers, di/tri/tetracarboxylic acid molecules such EDTA etc., hydrophilic and other D- and L-configurations of amino acids such as charged:

Arginine-Arg—R

Lysine—Lys—K (poly 1-lysine)

Aspartic acid—Asp—D

Glutamic acid—Glu—E

Polar amino acids (may participate in hydrogen bonds):

Glutamine—Gln—Q

Asparagine—Asn—N

Histidine—His—H

Serine—Ser—S

Threonine—Thr—T

Tyrosine—Tyr—Y

Cysteine—Cys—C

Methionine—Met—M

Tryptophan—Trp—W

Hydrophobic amino acids (normally buried inside the protein core):

Alanine—Ala—A

Isoleucine—Ile—I

Leucine—Leu—L

Phenvlalanine—Phe—F

Valine—Val—V

Proline—Pro—P

Glycine—Gly—G

poly-amino acid polymer (poly-1-lysine, poly histidine etc), and

Poly(vinylphosphonic acid).

Examples of crosslinkers that can modulate the adhesivity of the substrate include, but are not limited to, the following:
(1,4-cyclohexane dimethanol diglycidyl ether,
Neopentylglycol diglycidyl ether, 1,4-butanediol diglycidyl ether, ethylene glycol diglycidyl ether, polypropylene glycol
diglycidyl ether, resorcinol diglycidyl ether, glycerol diglycidyl ether, polyethylene glycol diglycidyl ether), polymethyl
methacrylate, polyethylene glycol methyl ether, polyethylene glycol diacrylate, polyethylene glycol diamine, Poly(2-hydroxyethyl methacrylate), Poly(D,L-lactide-co-glycolide), poly-lactic acid, poly-glycolic acid, Poly[(R)-3-hydroxybutyric acid], Poly(dimethylsiloxane), vinyl terminated, Poly(dimethylsiloxane), and diglycidyl ether terminated.
   The following claims are not intended to be limited to the embodiments, methods, and examples described herein.

1. A hydrogel composition, comprising a plurality of randomly alternating units of monomers crosslinked into a polymeric substrate with a crosslinker.

2. The composition of claim 1, wherein the monomers are selected from the group consisting of one or more of: streptomycin, neomycin, framycetin, paromomycin, ribostamycin, kanamycin, arbekacin, bekanamycin, dibekacin, tobramycin, spectinomycin, hygromycin b, gentamicin, netilmicin, sisomicin, isepamicin, verdamicin, amikacin, astromicin, apramycin, collagen, fibronectin, laminin, extracellular matrix, fibrin, short peptides, RGD peptide, polyethyleneimine,
oligonucleotides, aptamers, di/tri/tetracarboxylic acid, EDTA, arginine, lysine, aspartic acid, glutamic acid, glutamine, asparagine, histidine, serine, threonine, tyrosine, cysteine, methionine, tryptophan, alanine, isoleucine, leucine, phenylalanine, valine, proline, glycine, poly-amino acid polymer (poly-1-lysine, poly histidine) and Poly(vinylphosphonic acid).

3. The composition of claim 1 wherein the crosslinker is selected from the group consisting of one or more of: 1,4-cyclohexane dimethanol diglycidyl ether, Neopentylglycol diglycidyl ether, 1,4-butanediol diglycidyl ether, ethylene glycol diglycidyl ether, polypropylene glycol diglycidyl ether, resorcinol diglycidyl ether, glycerol diglycidyl ether, polyethylene glycol diglycidyl ether, polymethyl methacrylate, polyethylene glycol methyl ether, polyethylene glycol diacrylate, polyethylene glycol diamine, Poly(2-hydroxyethyl methacrylate), Poly(D,L-lactide-co-glycolide), poly-lactic acid, poly-glycolic acid, Poly[(R)-3-hydroxybutyric acid], Poly(dimethylsiloxane), vinyl terminated, Poly(dimethylsiloxane) and diglycidyl ether terminated.

4. The composition of claim 1, wherein the monomer is aminoglycoside amikacin AM1 or aminoglycoside amikacin AM3.

5. The composition of claim 1, wherein said hydrogel comprises aminoglycoside amikacin.

6. A method for generating a three dimensional (3D) dormant, relapsed and metastatic tumor microenvironment using the hydrogels composition of claim 1 comprising the steps of growing one or more cancer cells on said composition.

7. The method of claim 6, wherein said one or more cancer cells are co-cultured with fibroblast cells.

8. The method to claim 6, wherein one or more cancer cells is selected from the group consisting of T24 bladder cancer cells, UMUC3 bladder cancer cells, and NIH3T3-T24 co-culture 3DTM cells.

9. The method of claim 6, further comprising transferring said one or more cancer cells to a non-adhesive hydrogel comprising Amikacin AM3 to induce metastases and thereby forming metastatic cancer cells.

10. The method of claim 9, wherein said metastatic cancer cells are isolated from dormant cells by fluorescence activated cell sorting.

11. The method of claim 9, wherein an anticancer drug, biologic, immunotherapy or a combination thereof are added to said metastatic cancer cells to isolate resistant metastatic cells.

12. The method of claim 11, wherein said anticancer drug is docetaxel.
